(19) United States
(12) Patent Application Publication
(10) Pub. No.: US 2016/0250464 A1
(43) Pub. Date: Sep. 1, 2016

(54) DEVICE FOR THE APPLICATION OF A TRANSCUTANEOUS ELECTRIC STIMULATION STIMULUS

(71) Applicant: cerbomed GmbH, Erlangen (DE)

(72) Inventors: Thomas ZSCHAECK, Nuernberg (DE); Andreas HARTLEP, Holzkirchen (DE)

(21) Appl. No.: 15/052,075

(22) Filed: Feb. 24, 2016

(30) Foreign Application Priority Data
Feb. 27, 2015 (DE) 10 2015 002 589.9

(51) Int. Cl. A61N 1/36; A61N 1/04

(52) U.S. Cl. CPC A61N 1/36014; A61N 1/0456

(57) ABSTRACT

A device for the application of a transcutaneous electric stimulation stimulus onto the surface of a section of the human ear, includes at least two electrodes and a control device designed for input of a stimulation current via the electrodes. The stimulation device includes: a storage element that stores an allowed range of values for a contact resistance, which contact resistance is given between the at least two electrodes when the device is operated according to the intended use; a measuring device for measuring the contact resistance which is given between the at least two electrodes; a unit for changing the range of values for the contact resistance which range of values is stored in the storage element. The unit can change the stored range of values when a contact resistance measured by the measuring device lies outside of the stored allowed range of values.

### CROSS-REFERENCE TO RELATED APPLICATIONS

The present application claims priority of DE 10 2015 002 589.9, filed Feb. 27, 2015, the priority of this application is hereby claimed and this application is incorporated herein by reference.

### BACKGROUND OF THE INVENTION

The invention relates to a device for the application of a transcutaneous electric stimulation stimulus onto the surface of a section of the human ear which comprises at least two electrodes, wherein the device comprises a control device which is designed for an input of a stimulation current via the electrodes.

It is generally known to take influence on the neurophysiological and neuro-electrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

A device of the kind mentioned above is known from DE 10 2010 054 165 B3. Here, a device for the transcutaneous stimulation of the vagus nerve of the human body is described which electrode head with two electrodes is arranged in the region of the Cymba conchae; such a positioning of the electrodes has been proven as being beneficial. Thereby, the region of the Cymba conchae is that region of the concha of the ear which is located above the Crus helicis; it is also called Hemiconcha superior. Then, below the Crus helicis downwards the region of the Cavum conchae extends.

It is necessary for a satisfactory success of the therapy by application of a transcutaneous acting stimulation current to apply a sufficient intensity of current which of course at the other hand has to be not too high. In this connection it is problematic that the individual given contact resistance between the electrodes, which they have at intended use of the device, depends on the patient and is also dependent from further ambience parameters.

Namely, the skin resistance at different humans is different and can absolutely vary by the factor 10. A further influence value is the exact resting location of the electrodes. If for example an electrode rest directly on a perspiratory gland the skin resistance tends to low values; the skin resistance becomes still lower if the patient sweats (i. e. for example in the summer and at sunshine). Further other values for the skin resistance result at salty skin in the ear (e. g. in the case of dried sea water).

Accordingly a respective specific adaption of the stimulation current at the transcutaneous stimulation is laborious and difficult.

A stimulation device according to the generic type is also known from DE 10 2011 018 228 A1. A similar solution is known from US 2009/0082831 A1.

### SUMMARY OF THE INVENTION

Thus, it is an object of the present invention, to supply a device of the above mentioned kind by which it is possible to provide the respective optimal parameters for a transcutaneous stimulation which are patient specific as well as dependent from the ambience conditions. Accordingly, an automatic optimal adaption of the transcutaneous stimulation to an individual patient should be carried out.

The solution of this object by the invention is characterized in that the stimulation device comprises:
a storage element in which an allowed range of values for a contact resistance can be stored which contact resistance is given between the at least two electrodes when the device is operated according to the intended use;
a measuring device for measuring of the contact resistance which is given between the at least two electrodes when the device is operated according to the intended use;
means for changing of the range of values for the contact resistance which range of values is stored in the storage element, wherein the means can change the stored range of values when a contact resistance which is measured by the measuring device lies outside of the stored allowed range of values.

Especially, the mentioned means for changing of the range of values for the contact resistance which are stored in the storage element are designed to change the stored range of values only after an inquiry at the user when—as mentioned—a contact resistance which has been measured by the measuring device is outside of the stored allowed range of values.

The means for changing of the stored range of values for the contact resistance which range of values is stored in the storage element can thereby comprise a releasing element which can be actuated by the user of the device, wherein the means are designed to carry out a change of the stored range of values only when the releasing element was activated.

The device comprises preferably furthermore means for the output of a signal. The device can thereby be designed to activate those means for the output of a signal after a comparison was carried out between a contact resistance which was measured by the measuring device and a stored range of values for the contact resistance which range of values is stored in the storage element. Furthermore, it can be provided that the device is designed to output a warning signal via the output means for a signal when a contact resistance which was measured by the measuring device is outside of the stored range of values for the contact resistance which range of values is stored in the storage element. The device can furthermore be designed to output a releasing signal via the output means for a signal when a contact resistance which was measured by the measuring device is inside of the stored range of values for the contact resistance which range of values is stored in the storage element. The output means for a signal are thereby preferably a display or a screen. Alternatively or additively also an acoustic output element is possible.

The device can furthermore be designed to record values and to store the same in the storage element for supplying of an initial range of values for the contact resistance by means of the measuring device for measuring of the contact resistance, wherein the range of values is defined in that an excess value for exceeding of the stored maximum value is pretended and a underrun value for underrunning of the stored minimum value is pretended.

The device can furthermore be designed to record values and to store the same in the storage element for supplying of an actual individual range of values for the contact resistance by means of the measuring device for measuring of the contact resistance, wherein the range of values is defined in that an excess value for exceeding of the stored maximum value is pretended and a underrun value for underrunning of the stored minimum value is pretended, wherein especially a predetermined number of values which have been measured lastly are underlaid.

The excess value for exceeding is thereby preferably 20% of the stored maximum value; the underrun value for underrunning is preferably 20% of the stored minimum value.

The design of the proposed device is thus basing on the idea to give a message to a patient at a too high resistance (like for example “control the position of the ear electrode”) as well as at a too low resistance (for example “short circuit” —“short circuit” means here not a short circuit in the electro-technical meaning but that the resistance between the electrodes is extraordinary low).

If a contact resistance is determined via the measuring device which significantly differs from the stored allowed range of values the patent must confirm—via the releasing element—that he is stimulating in a right manner (he must feel a “prickling” due to the transcutaneous stimulation). The device stores then this new value in the storage element as correct and allows the changed allowed range of values at the next treatments.

For example as the minimum of the allowable range of values can be taken the lowest value minus an underrun value of 20% of 8 values (from stimulations of 2 days), while the highest value plus an excess value of 20% can be determined as maximum.

Underruns the patient at an application the minimum he must confirm the correct stimulation. For the excess of the maximum the same applies analogue. The oldest of those 8 values is continuously renewed at each completed stimulation period. If applicable, of course more than 8 lastly given values can be taken into account, for example 12 or 16 values (i. e. evaluation during 3 or 4 days).

Thereby, if applicable also a weighting of the measured values for the contact resistance can take place taking into account the respective duration of the stimulation. By doing so it can be reached that at very short stimulation sessions the resistance is not adjusted according to momentary values.

With the proposed solution it becomes thus possible to recognize failures in an easier manner like short circuits of the ear electrodes or a too high contact resistance. By the proposed design the stimulation device is enabled to register during some stimulation sessions the typical patent-specific skin resistance and to store it then as a patient-typical value and a patent-specific range of values respectively. At a significant difference from this value a warning (e. g. “Please confirm that the electrode is in a correct position”) or an error message can be issued.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows schematically the design of a stimulation device for the transcutaneous stimulation of a section of the human ear.

FIG. 2 shows schematically a number of measured values for the contact resistance between two electrodes of the device along the course of time.

### DETAILED DESCRIPTION OF THE INVENTION

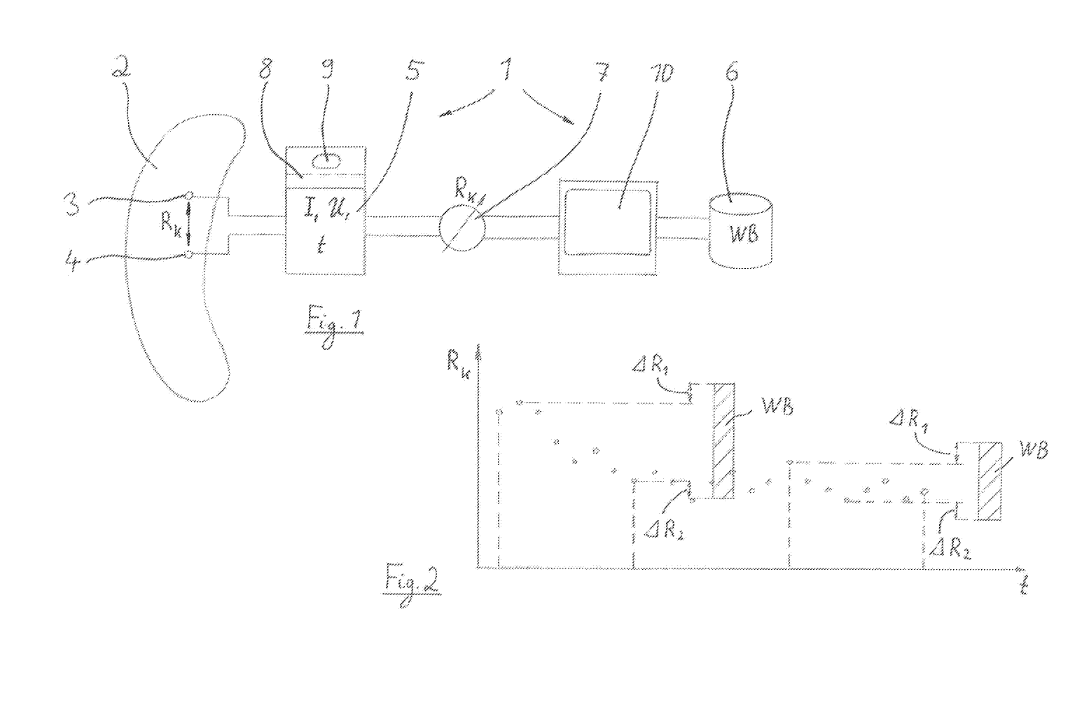

In FIG. 1 it is shown how a device 1 for the application of a stimulation stimulus is designed and which component it comprises respectively. To apply a transcutaneous stimulation stimulus on a section of the human ear 2 two electrodes 3 and 4 are provided which can be brought into skin contact with the ear 2. Such a stimulation device is known, wherein explicit reference is made to DE 10 2010 054 165 B3 of the patent proprietor is made where more specific explanations can be found. Accordingly, the device is designed to be arranged in the region of the vagus nerve at the ear of the user. Thereby, a transcutaneous stimulation of the vagus nerve can be carried out.

A control device 5 causes the charging of the electrodes 3, 4 with a stimulation current I at a given voltage U according to a predetermined time profile. It is essential that with the electrodes 3, 4 and the control device 5 also further components are in communication, namely a storage element 6 for storing of a range of values WB for allowable contact resistances R_{K}, a measuring device 7 for measuring of the contact resistance R_{K} and output means 10 for the issue of a signal, in the present case a display. Furthermore, means 8 for changing of the stored range of values WB are in communication with the arrangement, wherein at those means 8 also a releasing element 9 is arranged.

In FIG. 2 it is shown schematically how an allowable range of values WB for the contact resistance R_{K} can result along the time t in dependence of a plurality of measured values. Here, it should be noted that the single measurement points are contact resistances R_{K} which have been measured by means of the measuring device 7 at individual sessions at which a transcutaneous stimulation has been carried out. In the left half of FIG. 2 it can be seen that the contact resistances R_{K} are on a relatively high level by trend from which the allowable range of values WB results which is shown on the left side of FIG. 2. For doing so the last 8 measuring values have been regarded and the respective maximum and minimum has been determined. The allowable range of values WB results in that an excess value ΔR₁ is added to the maximum value of the contact resistance R_{K}, which is presently 20% of the determined maximum value; correspondingly, an underrun value ΔR₂ was subtracted from the minimum value of the contact resistance R_{K} which is also 20% of the determined minimum value.

As can be seen, during the run of the time the contact resistance R_{K} was reduced by trend, as can be seen from the right half of FIG. 2. This can be occurred for example due to a change of the weather conditions and that because of a higher temperature and the occurrence of perspiration now lower contact resistances are given. The range of values WB which is now given is determined in the same matter as described before (see the marked excess values and underrun values ΔR₁/ΔR₂).

In the storage element 6 the respective actual allowable range of values WB for the contact resistance R_{K} is stored. If the device 1 is intended used for the transcutaneous stimulation and if the measuring device 7 measures a value for the contact resistance R_{K} which is within the range of values WB the stimulation is carried out without further ado and namely without any issuance of a message to the user. Of course also a message can be issued that the transcutaneous stimulation occurs in good order.

However, if after the arrangement of the device at or in the ear 2 a contact resistance R_{K} is detected by means of the measuring device 7 which is outside of the stored actual range of values WB a respective message is given to the user via the display 10. Depending of the fact if the measured contact resistance R_{K} is too high or too low —respectively compared with the stored range of values WB—a corresponding message takes place.

Thus, if and in which manner a message occurs via the display 10 depends from the comparison of an actual measured contact resistance R_{K} with the actual stored range of values WB.

At a too high contact resistance R_{K}, which at a consequence demands a higher stimulation current I, the message can be: “Contact resistance too high, should the stimulation occur?”. If this is affirmed by the user, which can be signalized via the releasing element 9 of the device 1, the transcutaneous stimulation occurs, wherein the now used value of the contact resistance R_{K} is integrated in the determination of the allowed range of values WB.

At a too low contact resistance R_{K} the message can be: “Contact resistance too low, is the device inserted into the ear correctly?”. In this case namely a “short circuit” would be given which would inform the patient that he for example should clean the stimulation area prior the continuation of the treatment.

The management for the storage of the respective allowed range of values WB for the contact resistance R_{K} is done by the means 8 by which the range of values WB can be changed if this results from the above described procedure.

However, if from the user of the device no consideration of the new value of the contact resistance R_{K} should take place at a respective hint at the display he does not actuate the releasing element 9 so that the allowed range of values WB is not changed.

Prior to the first use of the stimulation device a typical allowed range of values WB for the contact resistance R_{K} can be factory-made stored in the storage element 6. The range of values WB can then—as explained above—be changed patent individually what can take place substantially automatically by the explained proceedings, i. e. without intervention by the physician or the patient.

While specific embodiments of the invention have been shown and described in detail to illustrate the inventive principles, it will be understood that the invention may be embodied otherwise without departing from such principles.

We claim:

1. A device for the application of a transcutaneous electric stimulation stimulus onto the surface of a section of the human ear which comprises at least two electrodes, wherein the device comprises a control device which is designed for an input of a stimulation current via the electrodes, wherein
that the stimulation device comprises:
a storage element in which an allowed range of values for a contact resistance can be stored which contact resistance is given between the at least two electrodes when the device is operated according to the intended use;
a measuring device for measuring of the contact resistance which is given between the at least two electrodes when the device is operated according to the intended use;
means for changing of the range of values for the contact resistance which range of values is stored in the storage element, wherein the means can change the stored range of values when a contact resistance which is measured by the measuring device lies outside of the stored allowed range of values.

2. The device according to claim 1, wherein the means for changing of the stored range of values for the contact resistance which range of values is stored in the storage element comprise a releasing element which can be actuated by the user of the device, wherein the means are designed to carry out a change of the stored range of values only when the releasing element was activated.

3. The device according to claim 1, wherein the device comprises furthermore means for the output of a signal.

4. The device according to claim 3, wherein the device is designed to activate the means for the output of a signal after a comparison was carried out between a contact resistance which was measured by the measuring device and a stored range of values for the contact resistance which range of values is stored in the storage element.

5. The device according to claim 4, wherein the device is designed to output a warning signal via the output means for a signal when a contact resistance which was measured by the measuring device is outside of the stored range of values for the contact resistance which range of values is stored in the storage element.

6. The device according to claim 4, wherein the device is designed to output a releasing signal via the output means for a signal when a contact resistance which was measured by the measuring device is inside of the stored range of values for the contact resistance which range of values is stored in the storage element.

7. The device according to claim 3, wherein the output means for a signal are a display or a screen.

8. The device according to claim 1, wherein the device is designed to record values and to store the same in the storage element for supplying of an initial range of values for the contact resistance by means of the measuring device for measuring of the contact resistance, wherein the range of values is defined in that an excess value for exceeding of the stored maximum value is pretended and a underrun value for underrunning of the stored minimum value is pretended.

9. The device according to claim 1, wherein the device is designed to record values and to store the same in the storage element for supplying of an actual individual range of values for the contact resistance by means of the measuring device for measuring of the contact resistance, wherein the range of values is defined in that an excess value for exceeding of the stored maximum value is pretended and a underrun value for underrunning of the stored minimum value is pretended, wherein especially a predetermined number of values which have been measured lastly are underlaid.

10. The device according to claim 8, wherein the excess value for exceeding is 20% of the stored maximum value and that the underrun value for underrunning is 20% of the stored minimum value.
